(19) United States
(12) Patent Application Publication
(10) Pub. No.: US 2017/0212292 A1
(43) Pub. Date: Jul. 27, 2017

(54) LIGHTING DEVICE FOR COUPLING LIGHT FROM A LIGHT SOURCE INTO A LIGHT GUIDE PLATE

(71) Applicant: PHILIPS LIGHTING HOLDING B.V., EINDHOVEN (NL)

(72) Inventor: Ype Bernardus BRADA, EINDHOVEN (NL)

(21) Appl. No.: 15/324,721

(22) PCT Filed: Jul. 8, 2015
(86) PCT No.: PCT/EP2015/065549

§ 371 Date: Jan. 8, 2017

(30) Foreign Application Priority Data
Jul. 8, 2014 (EP) 14176145.2

(51) Int. Cl. F21V 8/00; A61N 5/06

(52) U.S. Cl. CPC G02B 6/0031; G02B 6/0083; G02B 6/0085; A61N 5/06; A61N 2005/0663; A61N 2005/0651; A61N 2005/0665

(57) ABSTRACT

The present disclosure concerns a lighting device (100) comprising a light source (1) and a light guide plate (2). The light guide plate (2) is disposed at a distance (D1) from the emitter surface (1a) with an air gap (3) therebetween. A side reflector (4) surrounds the emitter surface (1a) of the light source and bounds the gap (3) between the emitter surface (1a) and the light entry surface (2a). The side reflector (4) comprises a tapered reflection surface (4a) adjacent the emitter surface (1a) and facing the light entry surface (2a). The tapered reflection surface (4a) is disposed at a tapering angle (β) towards the light entry surface (2a). The tapering angle (β) is configured such that any light emitted by the emitter surface (1a) within the opening angle (Φ) of the light source (1) is not reflected by the reflection surface (4a) before being received for the first time by the light entry surface (2a).

### TECHNICAL FIELD AND BACKGROUND

The present disclosure relates to a lighting device such as a light therapy device.

Light therapy consists of the exposure of a subject to a prescribed dosage of light. For example, light having a particular frequency may be used to treat circadian rhythm disorders. In the last decades, several types of light therapy devices have entered the market which typically comprise CFL lamps or LED lights. To improve eye comfort and freedom of design, these light sources can be combined with light guide plate technology. A light guide plate comprises a light entry surface at one or more edges of the light guide plate. The light entry surface is configured to receive incident light from the light source and transmit said light to an interior of the light guide plate. The interior is configured to guide and distribute the light over a (front) surface of the plate, e.g. via internal reflection and/or by reflective surfaces at the sides of the plate, except for the front surface where the light is emitted.

For the light therapy device to be effective, the lamps typically need a relatively high light output. In order to raise the output, it is preferred to use high power LEDs in combination with high output light guide plates. One way to couple the light from a flat-top LED into the plate is by mounting the LED in contact with the plate. However, working with high power LEDs can have additional problems. For example, the LEDs may become very hot, thereby degrading or even melting the material (typically plastic, more specifically PMMA) of the light guide plate. Furthermore, the LEDs can be sensitive to mechanical load. To alleviate such problems, the light guide plate can be disposed at a distance from the light source with an air gap therebetween. Also mechanical loading of the LED is prevented by creating a distance between LED and light guide plate. However, the gap may result in loss of light entering the plate and assembly problems for maintaining the distance between LED and light guide plate within a tolerance range.

To improve efficiency, the LED may be surrounded by a side reflector forming the edges of the gap. For example, US 2010/0271841 describes light emitting diodes at a side surface of a light guide plate and a side reflector on the side surface for reflecting light at the side surface back into the light guide plate, wherein the side reflector on the side surface has an opening corresponding to at least one of the light emitting diodes. For example, US 2014/0140091 describes a configuration which includes a strip of LEDs, wherein each LED is provided with an individual collimating element. For example, US 2013/0278612 describes a substantially etendue-preserving reflector between the light emitters and the light guide that can at least partially collimate light propagating in a single plane of collimation.

However, a drawback of the prior art light devices is that only light at a relatively small angle of incidence is directly projected into the light guide plate, causing loss of light and visible light spots at the entrée of the LED in the plate. In the case of high power LEDs this problem is even more relevant because the LEDs are spaced further apart and the spots can be more visible. Accordingly, it is desired to provide a high efficiency light coupling between a light source and light guide plate, wherein heat and mechanical load problems are alleviated while the occurrence of visible light spots at the light entrée of the light guide plate is diminished.

EP 1 659 427 A1 discloses a backlight unit including at least one light source emitting light, a light guide panel guiding proceeding of light incident from a side surface of the light guide panel, a light incident portion of the light guide panel protruding to be inclined with respect to the side surface of the light guide panel and having first and second light incident surfaces on which light is incident, and first and second guide members arranged to face the first and second light incident surfaces, respectively, and guiding the light emitted from the light source to be incident on each of the first and second light incident surfaces, wherein an air gap is formed between each of the first and second light incident surfaces and each of the first and second guide members.

### SUMMARY

A first aspect of the present disclosure provides a lighting device comprising a light source and a light guide plate. The light source comprises an emitter surface and an opening angle and is configured to emit light having a light distribution. The light guide plate comprises a front face surface through which, during use, light is emitted, a back face surface opposite to the front face surface (2c), and a light entry surface disposed at a side edge surface of the light guide plate between the front face surface and the back face surface and facing the emitter surface, wherein the light entry surface is configured to receive incident light at different angles of incidence emitted by the emitter surface and to transmit at least a first part of the received incident light through the light entry surface into an interior of the light guide plate, wherein the interior of the light guide plate is configured to guide the first part of the received incident light away from the light entry surface and distribute the light over the front face surface to be emitted therefrom; and wherein the light guide plate is disposed at a distance from the emitter surface with a gas-filled gap therebetween for diminishing heating of the light guide plate by the light source. The lighting device further comprises a side reflector, adjacent the emitter surface, wherein the side reflector comprises a tapered reflection surface adjacent the emitter surface and facing the light entry surface. According to the invention, the lighting device is characterized in that the side reflector forms an edge of the gas-filled gap between the emitter surface and the light entry surface, wherein a tapering angle of the tapered reflection surface with respect to the light entry surface is configured such that any light emitted by the emitter surface within the opening angle of the light source, before being received for the first time by the light entry surface, is not reflected by the reflection surface and is transmitted from the emitter surface to the light entry surface only via the gas-filled gap. In other words, the tapered reflection surface extends to the light entry surface with an opening angle outside the opening angle of the light source.

In a particular embodiment, wherein the tapered reflection surface extends as a flat surface from a position adjacent to the emitter surface up to the light entry surface, the property of the tapered reflection surface that it does not reflect any light emitted by the emitter surface within the opening angle of the light source before being received for the first time by the light entry surface is achieved by a tapering angle β<(180°−Φ)/2, wherein Φ is the opening angle of the light source.

By arranging the light entry surface of the light guide plate at a distance from the emitter surface with a gas-filled gap therebetween, heat and mechanical load problems can be alleviated. The gas-filled gap or light chamber can diminish heat transfer from the light source to the light guide plate e.g. because conduction and radiation are projected over a larger area. For example, instead of a very high heat density over the contact area, causing very high peak temperatures (e.g. >100 degrees centigrade), the heat flow is reduced by the distribution of the heat over the larger surface, induced by the gas-filled gap, resulting in lower peak temperatures. For example, the gap may be an air gap or may contain other gas.

By arranging a reflection surface adjacent the emitter surface, part of the light that does not directly enter the light guide can be reflected a second or further time towards the light entry surface. By extending the tapered reflection surface to the light entry surface, and providing it with an opening angle outside the opening angle of the light source, a majority of the initially emitted light first encounters the entry surface of the light guide plate, i.e. without obstruction by the adjacent tapered reflection surface.

At the same time, by providing the reflection surface at a tapering angle towards the light entry surface, light reflected by the light entry surface and incident on the tapered reflection surface can be redirected at a decreased angle of incidence the second time it hits the light entry surface. By decreasing the angle of incidence, the percentage of transmitted light can be increased. Accordingly, the percentage of light entering the light guide can be increased, after a round trip, with the tapered reflection surface compared to a non-tapered surface. By increasing the percentage of light entering the light guide for subsequent round trips, overall efficiency of the device can be improved.

Compared to a collimating (curved) surface, the tapered surface can provide a relatively wide distribution of light angles. By keeping a relatively wide distribution of light angles at the input interface (as opposed to a collimated beam), the occurrence of visible light spots at the light entrée of the light guide plate can be diminished. Furthermore, while a curved surface can be useful for reflecting light that directly originates from the light source towards the light entry surface, it is less suited for redirecting light already reflected off the light entry surface. In particular, a curved surface may actually bend such reflected light away from the normal of the light entry surface in a secondary reflection.

A degree of flatness of the tapered reflection surface may e.g. be quantified by a relatively large radius of curvature. For example, it is found desirable that the radius of curvature is at least ten times the distance between the emitter surface and the light entry surface, preferably at least twenty times this distance, more preferably at least thirty times this distance. A tapered reflection surface having a higher degree of flatness may improve efficiency and/or spread of light over the light entry surface. By extending the tapered reflection surface as a flat surface extending from a position adjacent the emitter surface up to the light entry surface, light rays can be redirected multiple times towards the light entry surface until virtually all light has entered the light guide plate. Furthermore, by extending the extended tapered reflection surface up to the light entry surface, a large emission angle of the light source can be covered. This coverage can be further increased in an embodiment wherein a distance between the light entry surface and an edge of the tapered reflection surface adjacent to the light source is equal to or larger than a distance between the light entry surface and the emitter surface.

The tapered reflection surface can provide particular improvement of the transmission of light with an initially large angle of incidence. In a particular embodiment, the tapered reflection surface is configured to receive a second part of the incident light reflected by the light entry surface, and to reflect the second part back towards the light entry surface, wherein, for incident light reflected by the light entry surface at an angle of incidence greater than the tapering angle, the redirected light is reflected towards the light entry surface at a smaller angle of incidence relative to the light entry surface than the angle of incidence of the incident light reflected by the light entry surface. In other words, when incident light hits the light entry surface at an angle of incidence greater than the tapering angle, at least a specular reflection of this incident light is bent by the tapered reflection surface towards a normal of the light entry surface. It is noted that also diffuse reflections may occur, but on average the effect may still cause bending of light towards the normal. By reducing the tapering angle between the tapered reflection surface and the light entry surface, a wider range of incidence angles can be redirected towards the normal. Accordingly, it is found desirable to provide a relatively small tapering angle, e.g. less than 30° (degrees plane angle), preferably less than 20°, more preferably less than 10°, or even less, e.g. 5°. On the other hand, the amount of redirection by the reflection surface can also be dependent on the tapering angle, with a larger tapering angle providing a larger redirection. Accordingly, it is found desirable to provide a tapering angle of more than 2°, preferably more than 5°. The tapering angle can also be chosen in relation to a desired size of the gas-filled gap.

In an embodiment, the tapering angle of the tapered reflection surface is configured such that, for each of the different angles of incidence (θ0) of the incident light greater than 45°, the second part (L2) of the incident light (L0) reflected by the light entry surface (2a) is reflected by the reflection surface (4a) and redirected towards the light entry surface (2a) at a reflected angle of incidence (θ2), wherein 0<θ2<θ0. For angles of incidence of the incident light greater than 45°, a substantial portion of the incident light will be reflected by the light entry surface of the light guide plate when it reaches the light entry surface for the first time. Because, in this embodiment, all the light emitted by the light source at an angle of incidence greater than 45° and reflected by the light entry surface is redirected towards the light entry surface by the tapered reflection surface at a smaller angle of incidence, the efficiency of coupling the light into the light guide plate is improved to an optimal extent.

By arranging the emitter surface between tapered reflection surfaces on either side, the emitted light travelling in either direction can be handled. By providing a relatively large opening angle between the two tapered reflection surfaces, a relatively small tapering angle can be provided on either side. At the same time, the large opening angle can accommodate a wide angle emitting light source. Accordingly, it is found desirable to provide an opening angle between the two tapered reflection surfaces in a range between 120 and 175°, preferably between 140 and 175°, or even between 160 and 175°.

By extending the gas-filled gap with a spacing between a side of the light source and an opposing part of the side reflector, heat transfer to the side reflector may be reduced. By surrounding the light source on all other sides with an gas-filled gap, an increased amount of produced heat can be conducted e.g. through a circuit board on which the light source is mounted. Heat drainage can be further improved by providing a circuit board with an integrated or separate heat sink. For example, the circuit board can be arranged between the side reflector and a heat sink plate. The heat sink plate can be configured such as to conduct a majority of the heat produced by the light source away from the circuit board, in particular when the light source is surrounded by air gaps on all sides other than the side where the circuit board is situated. It is noted that as the air stands still in the gaps, the air may not have a cooling effect. Convection of heat therefore plays a minimal role. On the other hand, radiation and conduction may have a larger effect on heat transfer. For example, the distance between the LED and the light guide plate or the light chambers may help in reducing the peak temperature due to a reduction in radiation density. The insulation capability of air may also help in the conduction part of the heat flow. For the radiation part, air does not help. So, creating gaps will have only limited effect on the direction of the heat flow, although the insulating effect will help. It will be appreciated that the present disclosure can provide a particular benefit for the use of a high power LED as the light source. While LEDs are considered quite efficient in terms of production of light, they may still produce a percentage (e.g. approx. 70%) of direct heat. The main problem with the application of high power LEDs is the high concentration of heat generated in a very small light source resulting in a very high heat flow around the LED, resulting in a very high temperature of the directly surrounding materials. For example, the presently proposed structures with the air gap and/or heat sink may need to dissipate an excess of more than 1 Watt of direct heat from a high power LED.

In addition to tapered reflection surfaces from two sides of the light source, the gas-filled gap can be bounded by upright reflection surfaces from the third and fourth sides of the light source. The upright reflection surfaces can be arranged substantially perpendicularly to the light entry surface, i.e. parallel to a face of the light guide plate. For example, the upright reflection surfaces can be perpendicular to the light entry surface, within 10° or less e.g. within 5°. It will be appreciated that the upright reflection surfaces allow the light chamber or gas-filled gap to be relatively thin, thus matching a relatively thin light guiding plate. By making a thickness of the gas-filled gap smaller than a thickness of the light guide plate, it can be ensured that the gas-filled gap is fully covered by the light entry surface, also taking into account manufacturing tolerances.

Advantageously, the present configuration may be applied for example in a light therapy device. Such a device is typically configured for providing a relatively high light intensity, e.g. quantified as more than 5000 LUX, at a distance of 20 centimetres from the front face surface of the light guide plate, or even higher, e.g. more than 7000 LUX at said position. It will be appreciated that the advantages regarding temperature reduction, light efficiency as well as diminishing the visibility of light spots can improve the overall suitability of the device.

### BRIEF DESCRIPTION OF DRAWINGS

These and other features, aspects, and advantages of the apparatus and systems of the present disclosure will become better understood from the following description, appended claims, and accompanying drawing, wherein:

FIG. 1A shows a cross sectional front view of an embodiment of a lighting device detailing a light source and light guide plate with an air gap therebetween;

FIG. 1B shows a cross sectional side view corresponding to FIG. 1A;

FIG. 2A shows a schematic first example of light rays reflecting between a light entry surface of the light guide plate and a tapered reflection surface of a side reflector;

FIG. 2B shows a schematic second example similar to FIG. 2A, but with a different path of the light rays;

FIG. 3A shows a polar plot of light emission angles for a typical LED light source;

FIG. 3B shows an example graph of reflection coefficient as a function of incidence angle;

FIG. 4A shows a cross sectional front view of an embodiment of a light therapy device as an example implementation of a lighting device;

FIG. 4B shows a zoomed-in detail of FIG. 4A;

FIG. 5A shows a cross sectional side view of a top part of a light therapy device;

FIG. 5B shows a cross sectional side view of a bottom part of a light therapy device;

FIGS. 6A-6C show perspective views of embodiments of light therapy devices.

### DESCRIPTION OF EMBODIMENTS

Unless otherwise defined, all terms (including technical and scientific terms) used herein have the same meaning as commonly understood by one of ordinary skill in the art to which this invention belongs as read in the context of the description and drawings. It will be further understood that terms, such as those defined in commonly used dictionaries, should be interpreted as having a meaning that is consistent with their meaning in the context of the relevant art and will not be interpreted in an idealized or overly formal sense unless expressly so defined herein. In some instances, detailed descriptions of well-known devices may be omitted so as not to obscure the description of the present systems. Terminology used for describing particular embodiments is not intended to be limiting of the invention. As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features. Likewise it will be understood that when a connection between structures or components is described, this connection may be established directly or through intermediate structures or components unless specified otherwise. All publications, patent applications, patents, and other references mentioned herein are incorporated by reference in their entirety. In case of conflict, the present specification, including definitions, will control.

In one aspect, the present disclosure provides encapsulating the LED in a tapered reflecting chamber, projecting the light in an efficient way into a light guide plate and maintaining a distance to the surface of the plate. The efficiency is measured to be the same compared to LEDs making direct contact with the light guide plate. The temperature of the light guide plate can be controlled e.g. by changing the distance from the LED to the plate.

It is noted that, for example, WO 2013/152234 describes an LED lighting device including an array of LED dies located on a substrate, a grid structure over the substrate forming an array of cavities to surround the LED dies in respective cavities, an optically reflective coating covering exposed regions of the substrate, and optically transparent plates placed over the cavities of the grid structure, allowing transmission of light produced by a respective LED die inside a respective cavity. However, the transparent plates of this prior art device do not function as light guide plates, in particular they do not comprise a light entry surface disposed at an edge of the light guide plate facing the emitter surface. Nor are the transparent plates configured to guide a majority of the light inside and along the plate surface. Instead, light is transmitted through the front and back faces of the transparent plates without being guided along the plate surface. Furthermore, because of the closeness of the LED to the transparent plates, the optically reflective coating is not configured to receive a second part of the incident light reflected off the light entry surface, and to redirect the second part back towards the light entry surface. Furthermore, the issue of preventing the occurrence of visible light spots at the light entrée of the light guide does not play a role.

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. The description of the exemplary embodiments is intended to be read in connection with the accompanying drawings, which are to be considered part of the entire written description. In the drawings, the absolute and relative sizes of systems, components, layers, and regions may be exaggerated for clarity. Embodiments may be described with reference to schematic and/or cross-section illustrations of possibly idealized embodiments and intermediate structures of the invention. In the description and drawings, like numbers refer to like elements throughout. Relative terms as well as derivatives thereof should be construed to refer to the orientation as then described or as shown in the drawing under discussion. These relative terms are for convenience of description and do not require that the system be constructed or operated in a particular orientation unless stated otherwise.

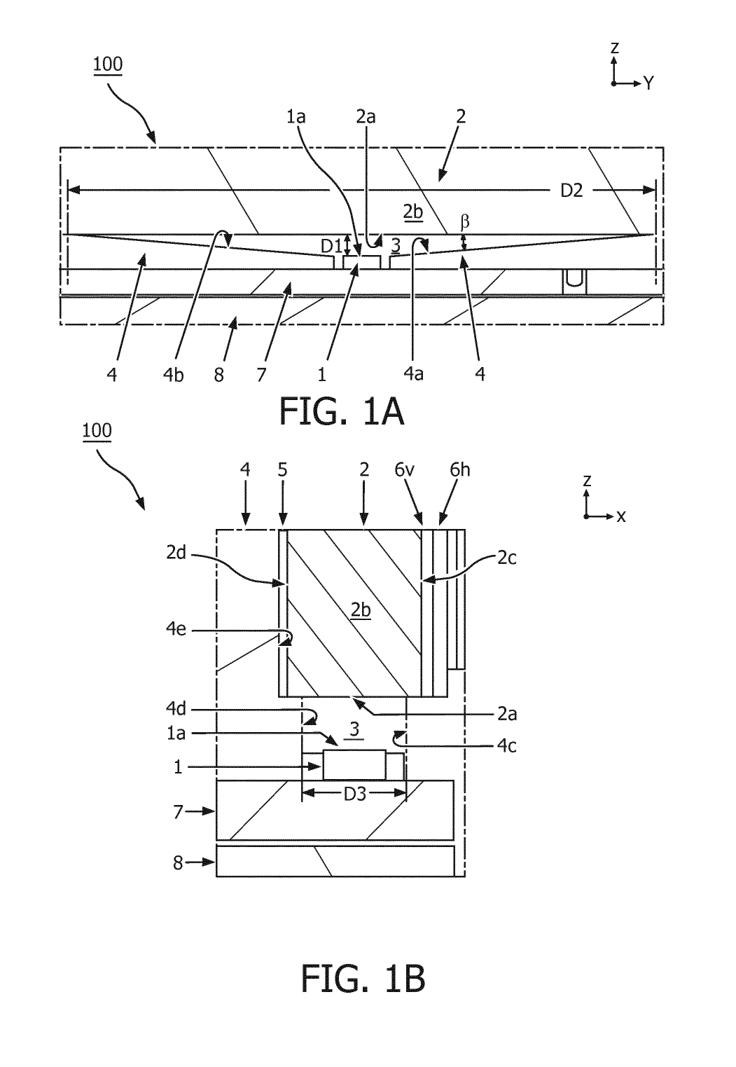

FIG. 1A shows a cross sectional front view (YZ) of an embodiment of a lighting device 100. FIG. 1B shows a cross sectional side view (XZ) corresponding to the embodiment of FIG. 1A.

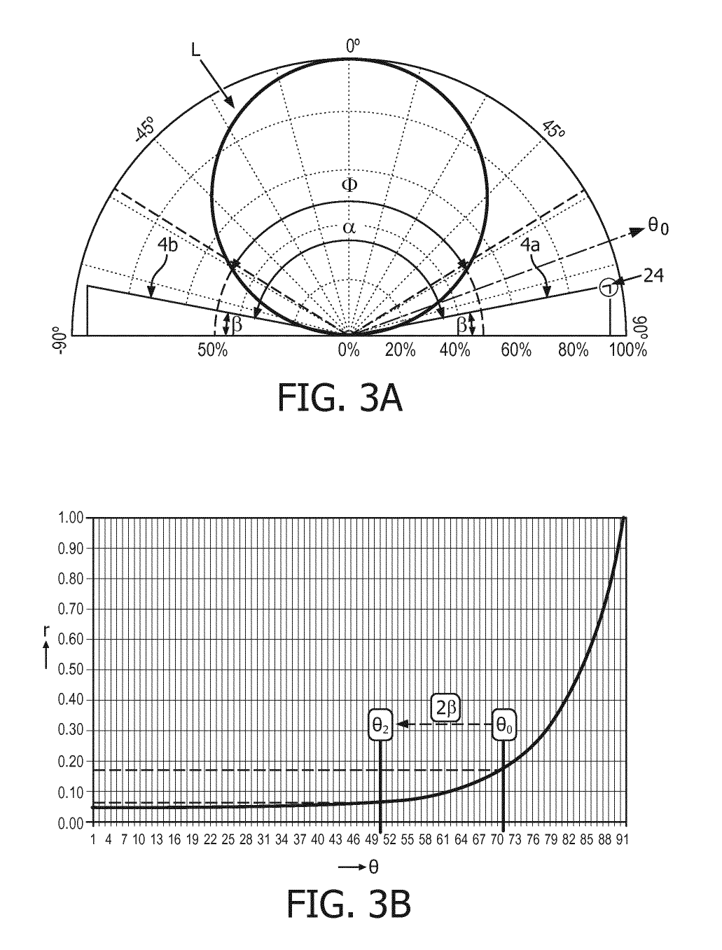

In one embodiment, the lighting device 100 comprises a light source 1. A light source is typically configured to emit a light distribution L through different angles. One way to characterize the spread of the light distribution is by means of the opening angle of the distribution. This will be further explained below with reference to FIG. 3A. It will be appreciated that the present disclosure provides particular benefit for light devices with a high power light source 1. Even an efficient high power light source, such as a high power LED, may produce direct heat in excess of 1 Watt together with the produced (useful) light. In one embodiment, the light source 1 is mounted on a circuit board 7. To prevent heat damage, preferably, the circuit board 7 is disposed between the side reflector 4 and a heat sink plate 8. For example, the heat sink plate 8 is configured to conduct heat produced by the light source 1 away from the circuit board 7. Alternatively, or in addition, the circuit board 7 may itself also function as a heat sink, e.g. a heat sink may be integrated in the circuit board 7. In one embodiment, the light source 1 comprises an emitter surface 1a configured to emit light. The emitter surface 1a may e.g. determine a direction in which most light is emitted. Preferably, the emitter surface 1a used in the present disclosure is substantially flat (a so-called flat-top LED) to efficiently direct the light, although also other shapes are possible.

In one embodiment, the lighting device 100 comprises a light guide plate 2. The light guide plate 2 comprises a light entry surface 2a disposed at a side edge surface of the light guide plate 2. The invention however is not limited to one side edge surface, e.g. light sources can be placed at multiple side edge surfaces. The light entry surface 2a is arranged to face the emitter surface 1a. The light entry surface 2a is configured to receive incident light from the emitter surface 1a and to transmit at least part of the received incident light through the light entry surface 2a into an interior 2b of the light guide plate 2. The interior 2b of the light guide plate 2 typically consists of a solid transparent body, e.g. comprising plastic or glass. In a light guide plate 2, light is predominantly transported inside the plate, e.g. by internal reflections. In particular, in the embodiment shown, the light guide plate 2 comprises a front face surface 2c and a back face surface 2d opposite to the front face surface 2c, wherein the front face surface 2c and the back face surface 2d are perpendicular to the light entry surface 2a. The interior 2b of the light guide plate 2 is configured to guide the transmitted part of the incident light from the light entry surface 2a and distribute the light over the front face surface 2c to be emitted therefrom.

In one embodiment, the light guide plate 2 is disposed at a distance D1 from the emitter surface 1a with an air gap 3 therebetween. The air gap 3, also referred to as light chamber, may help to alleviate, i.e. lower/diminish, local heating of the light guide plate 2 by the light source 1. The air gap 3 may e.g. comprise air or another gas. In one embodiment, the gap 3 comprises a substantially trapezoidal or near triangular shape. It will be noted that, in the embodiment shown, a width D2 of the gap 3 along a direction of the plate 2 is substantially larger than e.g. a height D1 between the emitter surface 1a and light entry surface 2a. This may correspond to a relatively small tapering angle β between the light entry surface 2a and the tapered reflection surface 4a.

The light guide plate 2 comprises a front face surface 2c through which light is emitted, a back face surface 2d opposite to the front face surface 2c, and a side edge surface 2a between the front and back face surfaces 2c, 2d. In one embodiment, the lighting device 100 comprises a back face reflector 4e on the back face surface 2d for reflecting light at the back face surface back into the light guide plate. In one embodiment, the lighting device 100 comprises light emitting diodes on the side edge surface 2a and a side reflector 4 on the side edge surface 2a for reflecting light incident on the side edge surface 2a back into the light guide plate. In one embodiment, the side reflector 4 on the side edge surface has an opening 3 corresponding to at least one of the light emitting diodes 1.

In one embodiment, the lighting device 100 comprises a side reflector 4. The side reflector 4 surrounds the emitter surface 1a. In addition, the side reflector 4 bounds, i.e. forms edges of, the air gap 3 between the emitter surface 1a and the light entry surface 2a. Also other surfaces may bound the air gap 3, e.g. part of the circuit board 7 on which the light source 1 is mounted. The side reflector 4 comprises a tapered reflection surface 4a adjacent, but preferably not connected to, the emitter surface 1a. The side reflector 4 faces the light entry surface 2a, i.e. a face of the side reflector 4 is directed towards the light entry surface 2a to be able to receive light therefrom. The tapered reflection surface 4a is disposed at a tapering angle β with respect to the light entry surface 2a. The direction of tapering is towards the light entry surface 2a, i.e. in a direction outwards from the light source 1. In one embodiment, the tapered reflection surface 4a extends to the light entry surface 2a with an opening angle α beyond the opening angle of the light source 1 (e.g. opening angle Φ shown in FIG. 3A).

In one embodiment, e.g. as shown in the cross sectional view YZ of FIG. 1A, the emitter surface 1a is arranged between two tapered reflection surfaces 4a, 4b on either side. At the same time, e.g. as shown in the cross sectional view XZ of FIG. 1B, the emitter surface 1a can be arranged between two upright reflection surfaces 4c, 4d of the side reflector 4 that are perpendicular to the light entry surface 2a. In one embodiment, e.g. as shown in the cross sectional view XZ of FIG. 1B, a width of the light guide plate 2 is larger than a thickness D3 of the air gap 3. In a further embodiment, a thickness of the light guide plate 2 extends on either side beyond the air gap 3 in the second cross sectional view XZ. In this way, manufacturing tolerances can be taken into account and it is guaranteed that all the generated light from emitter surface 1a is projected into the light entry surface 2a.

In one embodiment, e.g. as shown in FIG. 1B, the side reflector 4 is part of a larger body that extends to cover also a back side face 2d of the light guide plate 2. In particular, in the embodiment, the reflecting body 4 comprises a back reflector surface 4e configured to reflect light exiting the back side face 2d of the light guide plate 2. By having multiple reflecting surfaces integrated in a single reflector body, the construction may be simplified. Alternatively, it may be envisaged that each of the reflecting surfaces 4a-4e is part of separate reflecting bodies.

In one embodiment, a diffusor reflector foil 5 is arranged between the back reflector surface 4e and the backside face 2d of the light guide plate 2. The diffusor reflector 5 is not only configured for reflecting the reflected light but also for diffusing an angle of the reflected light. In this way, a more homogeneous light distribution may be achieved and the efficacy of the brightness enhancement foils 6v and 6h is improved. Alternatively, or in addition, also the back reflector surface 4e may have a diffusing effect on the reflected light angle. For example, the back reflector surface 4e and/or the diffusor reflector 5 may comprise a roughened surface to scatter the reflected light.

In one embodiment, brightness enhancement foils 6v,6h are arranged at a front side face 2c of the light guide plate 2. For example, in one embodiment, the foil 6v is configured to collimate outgoing light in a vertical direction while the foil 6h is configured to collimate outgoing light in a horizontal direction. The collimation may e.g. be effected by ridges on the foil. A combined effect of the foils may be that the brightness of the light is enhanced in particular directly in front of the output face of the device. Of course, the foils are optional, and may be omitted or replaced by other collimating structures.

In one embodiment, the air gap 3 has a width D2 and a thickness D3 measured in perpendicular directions Y,X along the light entry surface 2a of the light guide plate 2, wherein the width D2 is at least five times larger than the thickness D3. In this way a single light source may cover a relatively large part of a light guide plate 2 having itself a limited thickness.

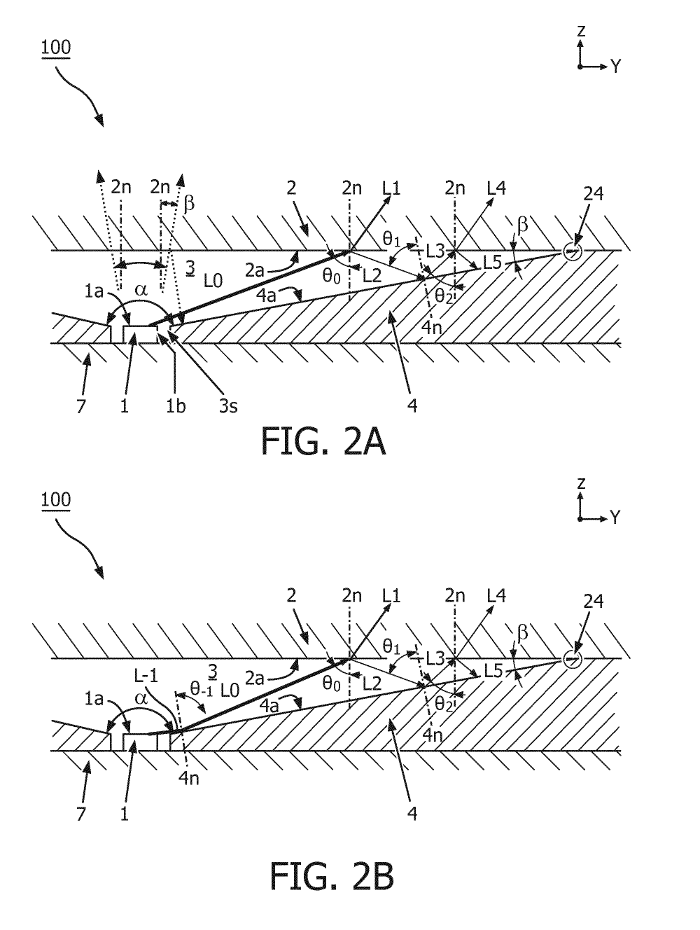

FIG. 2A shows a schematic first example of light rays reflecting between a light entry surface 2a of the light guide plate 2 and a tapered reflection surface 4a of a side reflector 4. FIG. 2B shows a schematic second example similar to FIG. 2A, but with a different path of the light rays.

In one embodiment, incident light L0 is received from the light source 1 at a light entry surface 2a of the light guide plate 2. A first part L1 of the incident light L0 is transmitted through the light entry surface 2a into an interior 2b of the light guide plate 2. A second part L2 of the incident light L0 is reflected off the light entry surface 2a. The side reflector 4 comprises a tapered reflection surface 4a. The tapered reflection surface 4a is configured for redirecting the second part L2 back towards the light entry surface 2a. In this example, the redirected light L3 is bent towards a normal 2n of the light entry surface 2a compared to the first-mentioned incident light L0 reflected off the light entry surface 2a.

Without wishing to be bound by theory, it may be deduced from geometrical considerations that the incidence angle θ2 of the redirected light L3 with respect to the normal 2n of the light entry surface 2a is changed with respect to the initial incidence angle θ0 by an amount of twice the tapering angle β, i.e. θ2=θ0−2β. Accordingly, it can be deduced that for incident light L0 reflected off the light entry surface 2a at an angle of incidence θ0 larger than the tapering angle β, the angle θ2 of the next reflection will be reduced, i.e. bent towards the normal. Because, in the present embodiment, the tapering angle β is relatively small, e.g. 10°, the majority of the redirected light L3 will have a smaller angle of incidence θ2 than the initial angle of incidence θ0. The effect of the total of transmitted light may be more pronounced because for small angles of incidence, the reflection L2 off the light entry surface 2a is already minimal. Furthermore, for geometrical considerations, the opening angle α between the tapered surfaces 4a,4b, may also be related to the tapering angle β as α=180−2β. Accordingly, it is preferred to provide a relatively large opening angle α between the two tapered reflection surfaces 4a, 4b.

In one embodiment, the light L2, L5, reflected off the light entry surface 2a of the light guide plate 2 is bent closer to a normal 2n of the light entry surface 2a of the light guide plate 2 at each subsequent specular reflection off the tapered reflection surface 4a. In one embodiment, the tapered reflection surface 4a extends from a position adjacent the emitter surface 1a towards the light entry surface 2a. The tapered reflection surface 4a is disposed at an angle β with respect to the light entry surface 2a. In the embodiment shown, the tapered reflection surface 4a extends as a flat surface between a position adjacent the emitter surface 1a and the light entry surface 2a, illustrated as intersection 24. Such an extension of the tapered reflection surface 4a can have the advantage that light can be reflected multiple times between the surfaces 2a and 4a without escaping from the gap 3. For example, if there is any remaining light L5 after the redirected light L3 has hit the input interface 2a, this may be redirected once again towards the light entry surface 2a. Alternatively, e.g. if the amount of the reflected light L5 is negligible, the tapered reflection surface 4a may be truncated.

In one embodiment, the air gap 3 extends with a spacing 3s between a side 1b of the light source 1 and an opposing part of the side reflector 4. In this way, heat transfer by conduction (and radiation) from the light source 1 to the side reflector 4 may be diminished. In one embodiment, the side 1b comprises a non-emitting surface of the light source 1. In this way, heat transfer by radiation (and conduction) from the light source 1 to the side reflector 4 may be diminished.

In one embodiment, a plane of the tapered reflection surface 4a, when extended to intersect a side 1b of the light source 1, lies at the same level as or just below the emitter surface 1a. In other words, a distance between the light entry surface 2a and an edge of the tapered reflection surface 4a adjacent to the light source 1 is equal to or larger than a distance between the light entry surface 2a and the emitter surface 1a. For example, a plane of the tapered reflection surface 4a may intersect at a position between 0 and 30 percent of the height of the light source 1 above the substrate 7, e.g. a circuit board, on which the light source 1 is mounted. When the intersection is at the same level as or below the emitter surface 1a, light rays emitted from the emitter surface 1a at a large angle may first hit the tapered reflection surface 4a. This is illustrated e.g. in FIG. 2B, wherein the light ray L₋₁ emitted at a large angle hits the tapered reflection surface 4a at an angle θ₋₁ with respect to the normal 4n of the tapered reflection surface 4a. It will be appreciated that the tapered reflection surface 4a redirects also this light ray towards the light entry surface 2a at an angle θ0 that is closer to the normal 2n of the light entry surface 2a than the originally emitted ray. On the other hand, if the emitter surface 1a is too far above the point of intersection, light rays propagating at large angles travel further outwards before meeting the surface 4a.

FIG. 3A shows a polar plot of light emission angles for a typical LED light source. For illustrative purposes, the present plot also shows preferred angles β of the tapered reflection surfaces 4a, 4b, which are also related to the opening angle α. In one embodiment, the tapered reflection surface 4a extends to the light entry surface 2a at an opening angle α outside the opening angle of the light source 1

The opening angle Φ (also called viewing angle) of a light source as used herein is defined, at a given distance from the light source, as the angle between the two emission directions on opposite sides of the 0° (normal) direction, at which, for the given distance, the emitted light intensity L (e.g. Watt per unit solid angle, or luminous flux per unit area) is 50 percent of its maximum intensity. The maximum intensity is usually attained in the 0° (normal) emission direction. In the present example, the opening angle Φ is about 114° (from −57° to +57°, wherein the plot is at 50 percent of its maximum intensity value). Other light sources may show different distributions.

In one example, the initial angle of incidence θ0 is 70°. After reflecting off the light entry surface of the light guide plate and off the tapered reflection surface, the angle of incidence θ2 is shifted by twice the tapering angle β, which in this example is 10°. Accordingly, the secondary angle of incidence θ2 is 50° (70−2*10).

It will be appreciated that to obtain, on the one hand, a high efficiency of light injection into the light guide plate and, on the other hand, avoid visible light spots, the tapering angle β may be selected in dependence on the opening angle Φ. In one embodiment, it is found preferable to choose a tapering angle β that is a fraction “f” that ranges between 0.05 and 0.15 times the opening angle Φ of the light source (as defined above). For example, if the opening angle Φ is 120° and the tapering angle is 10°, the fraction “f” is 1/12≈0.8. In one embodiment, an opening angle α of the tapered reflection surfaces 4a, 4b is more than an opening angle Φ of the light source. In one embodiment, the opening angle α of the tapered reflection surfaces 4a,4b extends such that said tapered reflection surfaces intersect the light guide plate (not shown here) at a point of intersection 24. In one embodiment, the tapering angle β and/or the opening angle α of the tapered reflection surface are configured such that any light emitted by the emitter surface 1a within the opening angle Φ of the light source 1 is not reflected by the reflection surface 4a before being received for the first time by the light entry surface 2a. In this embodiment, as shown in FIG. 2A, any light L0 emitted by the emitter surface 1a within the opening angle Φ of the light source 1, before being received for the first time by the light entry surface 2a, is transmitted from the emitter surface 1a to the light entry surface (2a) only via the air gap 3. In this way, a majority of the emitted light distribution L will initially hit the light entry surface 2a of the light guide plate and not be obstructed by the side reflectors 4a, 4b. In one embodiment, for a given opening angle Φ of the light source, the tapering angle β is smaller than (180°−Φ)/2. In one embodiment, the tapering angle β is constant over the whole tapered reflection surface 4a. In another embodiment, the tapering angle β varies over the reflection surface 4a. In one embodiment, the point of intersection 24 between the light entry surface 2a and the tapered reflection surface 4a is disposed outside an opening angle Φ of the light source.

FIG. 3B shows an example graph of reflection coefficient “r” as a function of incidence angle θ at the entrance of the light guide plate. Such a graph can e.g. be produced using Fresnel equations and the refractive indices of the gap and light guide material. The present example is based on the most commonly used light guide material: PMMA (n=1.49, n is refractive index) and air (n=1). The calculation is based on non-polarized light. It may be noted that the reflection coefficient is lower at lower angles of incidence.

As an example, there is shown a first light ray hitting the input interface at an angle of incidence of 70°. According to the graph, 17% of the first light will be reflected off the light entry surface and 83% will be transmitted into the interior of the light guide. The reflected part will be sent to the tapered reflection surface and redirected back to the light entry surface. The redirection by the tapered surface changes the angle of incidence θ2 of the secondary light to 50°. According to the graph, now only about 6% of the second light will be reflected off the light entry surface while 94% will be transmitted into the interior of the light guide. The overall transmission in this case is: 83%+94%*17%≈99%. By comparison, when use is made of a non-tapered side reflector, wherein after the second light ray only 83%+83%*17%≈97% would be transmitted, i.e. the loss is three times higher. It is noted that the losses may increase, e.g. due to absorption at each reflection from an interface. Accordingly, the fewer the reflections needed to transmit the light, the more efficient the coupling.

In order to use the maximum amount of light emitted by the LED, the opening angle α of the chamber in the side reflector is preferred to be at least equal to the opening angle of the LED itself. For LEDs used in light guide applications this angle typically is 120°. In this case it is ensured that most of the light is directly emitted into the light guide plate, with a minimum light loss caused by unnecessary reflection at surface 4a. At a small angle of incidence (up to 40°) between the light guide plate and a light ray from the LED, the amount of reflection at the light guide plate surface is around 4%. At an angle of incidence of 60° the reflection increases to 9°. At higher angles of the angle of incidence, the reflection increases rapidly, even up to 100% at an angle of incidence of 90°. In order to reuse the reflected light (i.e. the part of the light that has not entered the plate), the ray is reflected at the tapered mirrored surface of the side reflector and subsequently reflected at a more efficient angle back into the light guide plate (θ0→θ2).

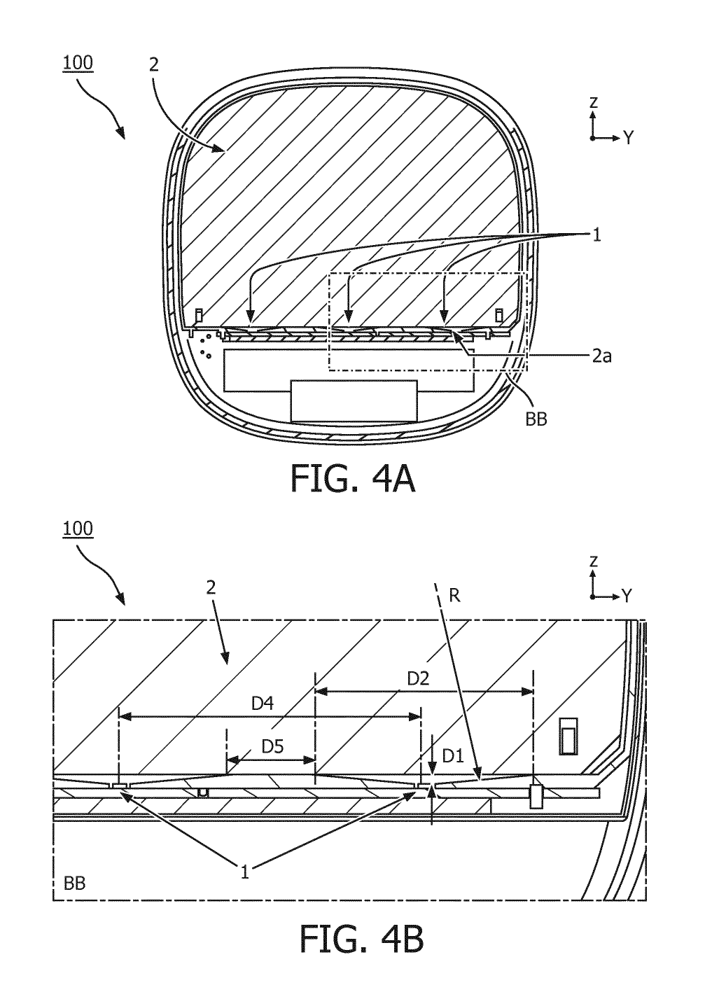

FIG. 4A shows a cross sectional front view of an embodiment of a light therapy device as an example implementation of a lighting device 100. In the figure, various parts of the lighting device 100 are visible, such as the light guide plate 2, and multiple light sources 1 at an input interface 2a of the light guide plate 2.

FIG. 4B shows a zoomed-in detail “BB” of FIG. 4A. In this detail, various dimensions D1-D5 of the device are shown. D1 indicates a distance between the emitter surface of the light source and the light entry surface of the light guide plate 2. D2 indicates a width of the gap. D4 indicates a distance between adjacent light sources. D5 indicates a distance between adjacent gaps. To achieve sufficient reduction of the heat flow density, preferably D1 is more than 0.5 mm, preferably more than 1 mm, or even more than 2 mm. The larger the distance D1, the better the temperature reduction on surface 2a. On the other hand, the light coupling efficiency may be diminished and/or may require too wide a gap. Accordingly, D1 is preferably less than 5 mm, more preferably less than 2 mm. To achieve an optimal spread and efficiency of light coupled into the plate 2, one or more of the following fractions are found to be preferable. A fraction D5/D4 is preferably between 0.1 and 0.4 to have sufficient coverage of the light guide plate. A fraction D1/D2 is preferably between 0.01 and 0.3 to provide a suitable tapering angle.

As noted above, a curved reflection surface may have as an undesirable effect that it limits the spread of the redirected beams. For example, a concave surface may lead to an increased degree of redirection of light having a larger initial angle. The light is thus more collimated by a curved surface, which may have as an undesirable effect that a visible light spot is produced at the interface with the light guide plate 2. Accordingly, in one embodiment, the tapered reflection surface 4a is substantially flat. The flatness can be quantified e.g. by a radius of curvature R. For example, in one embodiment, the radius of curvature R is at least ten times the distance D1 between the emitter surface 1a and light entry surface 2a, preferably at least twenty times said distance. Alternatively, or in addition, the radius of curvature may be indicated as an absolute number, e.g. preferably more than 10 mm, more preferably more than 100 mm, even more preferably, more than 1000 mm. This may be compared e.g. to a distance D1 of 1 mm.

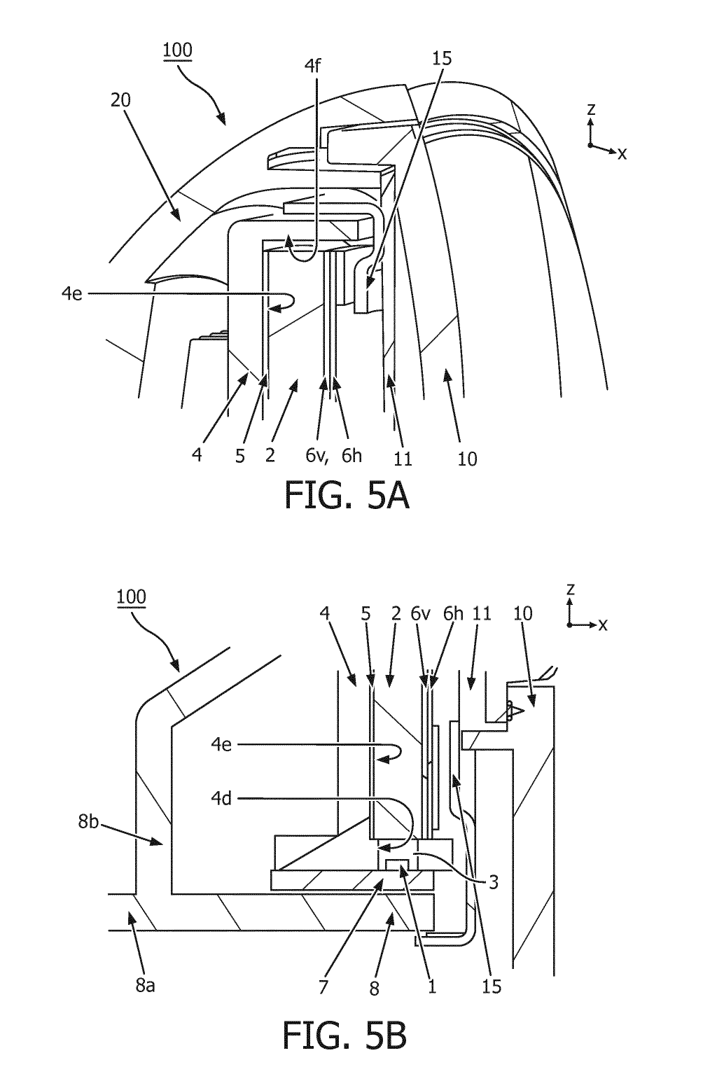

FIG. 5A shows a cross sectional side view (mostly XZ) of a top part of a light therapy device 100. FIG. 5B shows a cross sectional side view (XZ) of a bottom part of a light therapy device. In these figures, various components are visible that may be part of an embodiment of a light therapy device 100. Other embodiments may comprise additional or fewer components and/or in different arrangements and shapes.

For the present views of FIGS. 5A and 5B, the following components are indicated (from front to back): a front diffusing transmitter 10, a patterned foil 11, a clamp 15, brightness enhancement foils 6h and 6v, a light guide plate 2, a diffusor reflector foil 5, a reflector body 4, and a backside 20 of the device. In the shown embodiment, the reflector body encloses the light guide plate 2 on all sides, except the front face side and the positions of the light sources, where the air gap 3 is provided.

In the view of FIG. 5B, additionally the following components are indicated: a light source 1 disposed in a gap 3 in the side reflector 4, a circuit board 7, and a heat sink plate 8. In the embodiment shown, the heat sink plate 8 is configured to conduct heat away from the circuit board. To radiate the heat from the heat sink plate 8, it may comprise a back part 8b following a backside of the device 100.

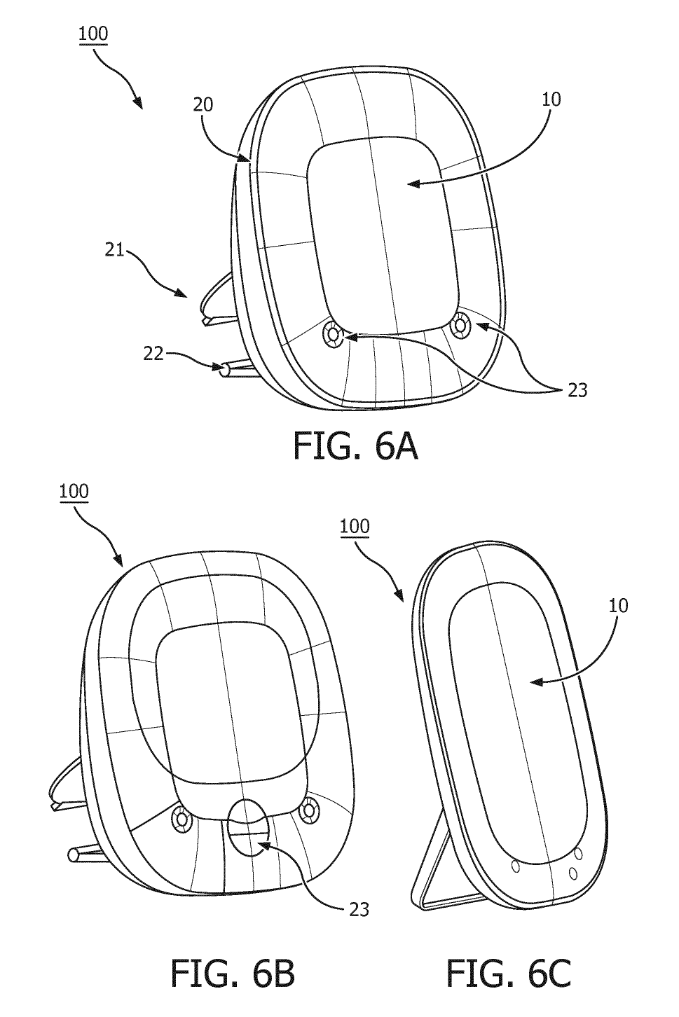

FIGS. 6A-6C show perspective views of embodiments of various light therapy devices. FIG. 6A shows an example of a high end device with blue LED light sources. The device 100 in this embodiment comprises a front 10, a back 20, a support foot 21, and an electricity wire. Of course also other configurations are possible. FIG. 6B shows another example of a device with blue LED light sources. The devices differ e.g. in the means of control 23. FIG. 6C shows yet another example of a light therapy device, in this case comprising white light LEDs. This device may e.g. have a larger front surface. Of course many different design variations are possible providing equivalent function. In general, it is preferred that a light therapy device be capable of delivering a certain dose of light. To ensure effective therapy, preferably, at least part of the light is in a blue part of the spectrum. In one embodiment, a lighting device is configured for providing a light intensity of more than 5000 LUX at a distance of 20 centimetres from the front face 10 of the device. For some applications, even higher intensities may be desired, e.g. more than 10000 LUX at the said distance.

For certain light therapy applications, it is found that light in a particular frequency range can be more effective than white light. For example, a total light intensity for achieving a desired effect can be lower when using only blue light. In one embodiment, a lighting device as described herein is configured to provide light in a wavelength range between 460-490 nanometres. In a further embodiment, a lighting device as described herein is configured to provide light in the said wavelength range between 460-490 nanometres, wherein the light intensity is more than 100 LUX at a distance of 50 centimetres from the front side 31 of the lighting device 100. Preferably, the light intensity is more than 200 LUX at the said distance and in the said wavelength range between 460-490. It is noted that LUX is a photometric measure of the intensity, as perceived by the human eye, i.e. power at each wavelength weighted according to the luminosity function.

For the purpose of clarity and a concise description, features are described herein as part of the same or separate embodiments, however, it will be appreciated that the scope of the invention may include embodiments having combinations of all or some of the features described. For example, while lighting device embodiments were shown with certain arrangements or combinations of components, also alternative ways may be envisaged by those skilled in the art having the benefit of the present disclosure for achieving a similar function and result. For example, optical and/or electrical components may be combined or split up into one or more alternative components. The various elements of the embodiments as discussed and shown offer certain advantages, such as alleviating heat transfer and efficient light distribution over a light guide plate. Of course, it is to be appreciated that any one of the above embodiments or processes may be combined with one or more other embodiments or processes to provide even further improvements in finding and matching designs and advantages. It will be appreciated that this disclosure offers particular advantages to light therapy devices, and in general can be applied for any application wherein high power light sources are used in combination with light guiding plates. But this disclosure is not limited to high power LEDs. The principle applies to all types of LEDs, even to other light sources. For example, in one embodiment, the present teachings may also be applied for providing back lighting to an LCD or other screen, e.g. television monitor.

While the present systems have been described in particular detail with reference to specific exemplary embodiments thereof, it should also be appreciated that numerous modifications and alternative embodiments may be devised by those of ordinary skill in the art without departing from the scope of the present disclosure. Finally, the above discussion is intended to be merely illustrative of the present systems and should not be construed as limiting the appended claims to any particular embodiment or group of embodiments. The specification and drawings are accordingly to be regarded in an illustrative manner and are not intended to limit the scope of the appended claims. In interpreting the appended claims, it should be understood that the word “comprising” does not exclude the presence of elements or acts other than those listed in a given claim; the word “a” or “an” preceding an element does not exclude the presence of a plurality of such elements; any reference signs in the claims do not limit their scope; several “means” may be represented by the same or different item(s) or implemented structure or function; any of the disclosed devices or portions thereof may be combined together or separated into further portions unless specifically stated otherwise. The mere fact that certain dimensions are recited in mutually different claims does not indicate that a combination of these dimensions cannot be used to advantage. In particular, all possible combinations of the claims are considered inherently disclosed.

1. A lighting device comprising
a light source comprising an emitter surface and an opening angle configured to emit light having a light distribution;
a light guide plate comprising a front face surface through which, during use, light is emitted, a back face surface opposite to the front face surface, and a light entry surface disposed at a side edge surface of the light guide plate between the front face surface and the back face surface and facing the emitter surface, wherein the light entry surface is configured to receive incident light at different angles of incidence emitted by the emitter surface and to transmit at least a first part of the received incident light through the light entry surface into an interior of the light guide plate, wherein the interior of the light guide plate is configured to guide the first part of the received incident light away from the light entry surface and distribute the light over the front face surface to be emitted therefrom; and wherein the light guide plate is disposed at a distance from the emitter surface with a gas-filled gap therebetween for diminishing heating of the light guide plate by the light source; and
a side reflector, adjacent the emitter surface, wherein the side reflector comprises a tapered reflection surface adjacent the emitter surface and facing the light entry surface,
characterized in that the side reflector forms an edge of the gas-filled gap between the emitter surface and the light entry surface, wherein a tapering angle of the tapered reflection surface with respect to the light entry surface is configured such that any light emitted by the emitter surface within the opening angle of the light source, before being received for the first time by the light entry surface, is not reflected by the reflection surface and is transmitted from the emitter surface to the light entry surface only via the gas-filled gap.

2. The lighting device according to claim 1, wherein the tapered reflection surface is configured to receive a second part of the incident light reflected by the light entry surface, and to reflect the second part back towards the light entry surface, wherein, for incident light reflected by the light entry surface at an angle of incidence greater than the tapering angle, the redirected light is reflected towards the light entry surface at a smaller angle of incidence relative to the light entry surface than the angle of incidence of the incident light reflected by the light entry surface.

3. The lighting device according to claim 1, wherein the tapering angle is configured such that, for each of the different angles of incidence greater than 45°, the second part of the incident light reflected by the light entry surface is reflected by the reflection surface and redirected towards the light entry surface at a reflected angle of incidence, wherein 0<θ2<θ0.

4. The lighting device according to claim 1, wherein the tapered reflection surface extends as a flat surface from a position adjacent the emitter surface up to the light entry surface, wherein the tapering angle β<(182°−Φ)/2.

5. The lighting device according to claim 1, wherein the gas-filled gap includes a spacing between a non-emitting surface of the light source and an opposing part of the side reflector.

6. The lighting device according to claim 1, wherein a distance between the light entry surface and an edge of the tapered reflection surface adjacent to the light source is equal to or larger than a distance between the light entry surface and the emitter surface.

7. The lighting device according to claim 1, wherein, in a first cross sectional view of the gas-filled gap perpendicular to the light entry surface, the emitter surface is arranged between two tapered reflection surfaces disposed on either side of the emitter surface, wherein an opening angle between the two tapered reflection surfaces is between 120 and 176°.

8. The lighting device according to claim 1, wherein, in a second cross sectional view of the gas-filled gap, perpendicular to the light entry surface, the emitter surface is arranged between two upright reflection surfaces of the side reflector that are perpendicular to the light entry surface or substantially perpendicular to the light entry surface within 10°.

9. The lighting device according to claim 1, wherein, in a thickness direction of the light guide plate, the light guide plate extends on either side of the gas-filled gap.

10. The lighting device according to claim 1, wherein the tapered reflection surface has a radius of curvature which is at least ten times the distance between the emitter surface and the light entry surface.

11. The lighting device according to claim 1, wherein the light source comprises a high-power LED which, in operation of the lighting device, produces direct heat in excess of 1 Watt.

12. The lighting device according to claim 1, wherein the light source is mounted on a circuit board, wherein the circuit board is disposed between the side reflector and a heat sink plate, wherein the heat sink plate is configured to transmit heat produced by the light source away from the circuit board.

13. The lighting device according to claim 1, wherein the front face surface of the light guide plate is perpendicular to the light entry surface.

14. A light therapy device comprising a lighting device according to claim 1, wherein the lighting device is configured to provide a light intensity in a wavelength range between 460-490 nanometres of more than 200 LUX at a distance of 50 centimetres from the front face surface of the light guide plate.
